SEQUENCE LISTING (1 sequence)

SEQ ID NO: 1 - protein, 585 aa, synthetic construct [sequence omitted]

(19) United States
(12) Patent Application Publication
(10) Pub. No.: US 2025/0242040 A1
(43) Pub. Date: Jul. 31, 2025

(54) ALBUMIN CONJUGATE, PREPARATION METHOD THEREFOR AND USE THEREOF

(71) Applicant: Suzhou KangDeRui Pharmaceutical, Ltd., Suzhou (CN)

(72) Inventors: Chengjie LIU, Suzhou (CN); Xiaodong LIU, Suzhou (CN); Zhenwei ZHANG, Suzhou (CN); Enze LIU, Suzhou (CN)

(21) Appl. No.: 19/031,048

(22) Filed: Jan. 17, 2025

Related U.S. Application Data
(63) Continuation of application No. PCT/CN2024/079217, filed on Feb. 29, 2024.

(30) Foreign Application Priority Data
Mar. 2, 2023 (CN) 202310264655.9

(51) Int. Cl. A61K 47/64; A61K 47/65; A61P 35/00

(52) U.S. Cl. CPC A61K 47/643; A61K 47/65; A61P 35/00

(57) ABSTRACT

Tumor-targeted therapeutic agents, and in particular an albumin conjugate, a preparation method therefor and use thereof in treating a tumor. The human serum albumin-conjugated drug can be recognized and internalized by tumor cells, and the therapeutic drug can be accurately delivered into the tumor, thereby enhancing the targeting effect of the drug and avoiding the indiscriminate killing on normal tissues, organs and cells by the drug.

### CROSS-REFERENCE TO RELATED APPLICATIONS

The present disclosure is a Continuation Application of International Application No. PCT/CN2024/079217, filed on Feb. 29, 2024, which claims priority to Chinese Patent Application No. 202310264655.9, entitled “ALBUMIN CONJUGATE, PREPARATION METHOD THEREFOR AND USE THEREOF”, filed with the China National Intellectual Property Administration on Mar. 2, 2023, the disclosures of both of which are hereby incorporated by reference in their entirety.

### CROSS-REFERENCE TO RELATED APPLICATIONS

The contents of the electronic sequence listing (2025-04-22-Sequence-Listing.xml; Size: 2,643 bytes; and Date of Creation: Mar. 31, 2025) is herein incorporated by reference in its entirety.

### TECHNICAL FIELD

The present disclosure relates to the field of tumor-targeted therapeutic agents, and in particular to an albumin conjugate, a preparation method therefor and use thereof in treating a tumor.

### BACKGROUND

Human serum albumin (HSA) is a kind of multifunctional blood protein that accounts for 35-55% of plasma proteins and has a molecular weight of 66.5 KD. It is very stable in different solvents over an extensive range of pH and temperature, and can be stored in dissolved form for a long period of time. HSA contains 585 amino acid residues, on which therapeutic molecules or signal groups for diagnosis can be linked.

Due to the vigorous metabolism of tumor tissues, solid tumors require a large amount of amino acids and energy during their growth, and albumin is the main source of amino acids and energy for solid tumors, so a large number of nutrient transporters (e.g., Gp18, Gp30, and Gp60 and secreted protein, acidic and rich in cysteine (SPARC), GLUT-1, LAT, LDL receptors, mannose receptors) would be expressed on the surface of tumor cells to meet the demands of growth.

For some tumor tissues, the therapeutic efficacy of a drug is mainly determined by two factors, the targeting ability and the penetration ability of the drug. Human serum albumin-conjugated therapeutic drug modified with anti-tumor peptides can be recognized and internalized by tumor cells, achieving the precise delivery ability and high permeability of the drug.

### SUMMARY

### Problems to be Solved

Antibody-drug conjugates (ADCs) constitute the main platform currently used for targeted drug delivery. ADCs have shown significant clinical success in treating hematologic tumors, but there are still issues in several aspects: 1. Antibodies in the antibody-drug conjugates are immunogenic and therefore can easily cause severe side effects such as interstitial pneumonia, myocarditis, gastroenteritis, hepatitis, and nephritis. These side effects caused by immune stimulation are not easy to be detected, and progress rapidly, and are very likely to cause death of patients. 2. Since targets of the antibodies are also expressed in normal cells, the antibody-drug conjugates are prone to cause indiscriminate killing of normal cells. 3. Tumor cells are prone to shield antibodies, resulting in immune escape. 4. Antibodies are internalized after binding to specific targets, which determines a single pathway for the drug to be internalized by the cell, resulting in a very low penetration rate.

### Technical Solutions

In one aspect, the present disclosure provides an albumin conjugate, or a pharmaceutically acceptable salt or a solvate thereof, where the albumin conjugate includes albumin and HcyTFAc,
the albumin is selected from a group consisting of:
a) a protein including an amino acid sequence as set forth in SEQ ID NO:1, preferably a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1;
the HcyTFAc has a structure of

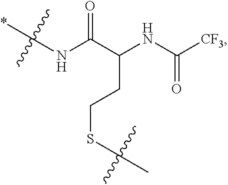

where * is a site directly linked to the albumin.

Preferably, the albumin conjugate includes a toxin molecule.

Preferably, the toxin molecule is selected from a group consisting of an antitubulin agent, maytansine, paclitaxel, camptothecin, duocarmycin, pyrrolobenzodiazepine (PBD), Eribulin, adriamycin, doxorubicin, gemcitabine, amethopterin, fluorouracil, Irinotecan, taxol, bleomycin, mitomycin, cytarabine, ramycin, vinblastine, vincristine, morpholine-adriamycin, trifluorothymidine, Dxd, a nucleic acid, a nuclide, and a metal.

Preferably, the toxin molecule is selected from a group consisting of the antitubulin agent, the maytansine, the paclitaxel, the camptothecin, the duocarmycin, the pyrrolobenzodiazepine (PBD), the Eribulin, the adriamycin, the Irinotecan, the Dxd, the nucleic acid, the nuclide, and the metal.

Preferably, the antitubulin agent is selected from a group consisting of a tubulin inhibitor.

Preferably, the antitubulin agent is selected from auristatin.

Preferably, the antitubulin agent is selected from auristatin E, auristatin F, and auristatin D.

Preferably, the antitubulin agent is selected from monomethyl auristatin E (MMAE), monomethyl auristatin F (MMAF), and monomethyl auristatin D (MMAD).

Preferably, the albumin conjugate includes a linker.

Preferably, the linker is selected from a group consisting of a thioether bond, a disulfide bond, an aminoamide bond, an amide bond, a peptide bond, and

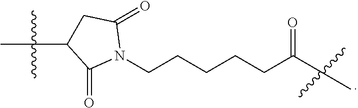 [SMILES: *C(=O)CCCCCN1C(=O)CC(*)C1=O]

Preferably, the linker is selected from a group consisting of the disulfide bond, the aminoamide bond, the amide bond, and the

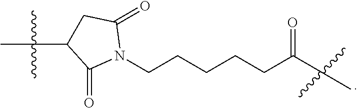 [SMILES: *C(=O)CCCCCN1C(=O)CC(*)C1=O]

Preferably, the linker is selected from the

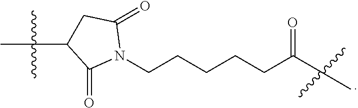 [SMILES: *C(=O)CCCCCN1C(=O)CC(*)C1=O]

Preferably, the albumin conjugate includes a linker unit.

Preferably, the linker unit is selected from a group consisting of an enzyme-cleavable linker unit and an enzyme-uncleavable linker unit.

Preferably, the enzyme-cleavable linker unit is selected from a group consisting of a protease cleavable linker unit.

Preferably, the protease cleavable linker unit includes a combination of two or more selected from a group consisting of valine, citrulline, alanine, glycine, lysine, phenylalanine, glutamic acid, serine, and aspartic acid.

Preferably, the protease cleavable linker unit includes a combination of one or more selected from a group consisting of valine-citrullinedipeptide, valine-alaninedipeptide, and glycine-glycine-phenylalanine-glycinetetrapeptide.

Preferably, the protease cleavable linker unit is selected from a group consisting of valine-citrulline-p-aminobenzyloxy (Val-Cit-PAB), valine-alanine-p-aminobenzyloxy (Val-Ala-PAB), glycine-glycine-phenylalanine-glycine (Gly-Gly-Phe-Gly), valine-citrulline-p-aminobenzyloxy (Val-Cit-PAB), valine-alanine-p-aminobenzyloxy (Val-Ala-PAB), and glycine-glycine-phenylalanine-glycine (Gly-Gly-Phe-Gly).

Preferably, the albumin conjugate has a structure as shown in Formula I:

HSA-(R-linker-linker unit-D)ₙ (Formula I)
where:
HSA is selected from a group consisting of:
a) a protein including an amino acid sequence as set forth in SEQ ID NO:1, preferably a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1;
R is selected from HcyTFAc;
D is selected from a toxin molecule;
where the HTLTFAc has a structure of

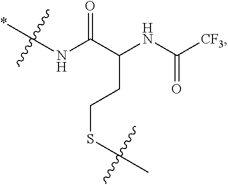

where * is a site directly linked to the albumin;
n is an integer selected from 1-16, preferably an integer selected from 2-8, more preferably an integer selected from 2-5.

Preferably, the toxin molecule in Formula I is selected from a group consisting of an antitubulin agent, maytansine, paclitaxel, camptothecin, duocarmycin, pyrrolobenzodiazepine (PBD), Eribulin, adriamycin, doxorubicin, gemcitabine, amethopterin, fluorouracil, taxol, bleomycin, mitomycin, cytarabine, ramycin, vinblastine, vincristine, morpholine-adriamycin, trifluorothymidine, Dxd, a nucleic acid, a nuclide, and a metal.

Preferably, the toxin molecule is selected from a group consisting of the antitubulin agent, the maytansine, the paclitaxel, the camptothecin, the duocarmycin, the pyrrolobenzodiazepine (PBD), the Eribulin, the adriamycin, the irinotecan, the Dxd, the nucleic acid, the nuclide, and the metal.

Preferably, the antitubulin agent is selected from a group consisting of a tubulin inhibitor.

Preferably, the antitubulin agent is selected from auristatin.

Preferably, the antitubulin agent is selected from auristatin E, auristatin F, and auristatin D.

Preferably, the antitubulin agent is selected from monomethyl auristatin E (MMAE), monomethyl auristatin F (MMAF), and monomethyl auristatin D (MMAD).

Preferably, the albumin conjugate includes a linker.

Preferably, the linker is selected from a group consisting of a thioether bond, a disulfide bond, an aminoamide bond, an amide bond, a peptide bond, and

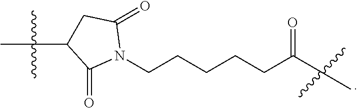 [SMILES: *C(=O)CCCCCN1C(=O)CC(*)C1=O]

Preferably, the linker is selected from a group consisting of the disulfide bond, the aminoamide bond, the amide bond, and the

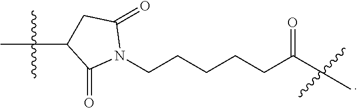 [SMILES: *C(=O)CCCCCN1C(=O)CC(*)C1=O]

Preferably, the linker is selected from the

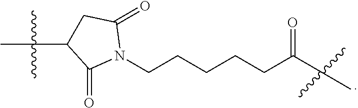 [SMILES: *C(=O)CCCCCN1C(=O)CC(*)C1=O]

Preferably, the linker unit is selected from a group consisting of an enzyme-cleavable linker unit.

Preferably, the enzyme-cleavable linker unit is selected from a group consisting of a protease cleavable linker unit.

Preferably, the protease cleavable linker unit includes a combination of two or more selected from a group consisting of valine, citrulline, alanine, glycine, lysine, phenylalanine, glutamic acid, serine, and aspartic acid.

Preferably, the protease cleavable linker unit includes a combination of one or more selected from a group consisting of valine-citrullinedipeptide, valine-alaninedipeptide, and glycine-glycine-phenylalanine-glycinetetrapeptide.

Preferably, the protease cleavable linker unit is selected from a group consisting of valine-citrulline-p-aminobenzyloxy (Val-Cit-PAB), valine-alanine-p-aminobenzyloxy (Val-Ala-PAB), glycine-glycine-phenylalanine-glycine (Gly-Gly-Phe-Gly), valine-citrulline-p-aminobenzyloxy (Val-Cit-PAB), valine-alanine-p-aminobenzyloxy (Val-Ala-PAB), and glycine-glycine-phenylalanine-glycine (Gly-Gly-Phe-Gly).

In another aspect, the present disclosure provides a pharmaceutical composition, including a therapeutically effective amount of the albumin conjugate, or the pharmaceutically acceptable salt or the solvate thereof, and one or more pharmaceutically acceptable carriers, diluents or excipients.

In another aspect, the present disclosure provides use of the albumin conjugate, or the pharmaceutically acceptable salt or the solvate thereof or the composition for preventing and/or treating a cancer.

In another aspect, the present disclosure provides use of the albumin conjugate, or the pharmaceutically acceptable salt or the solvate thereof or the composition in preparation of a drug for preventing and/or treating a cancer.

### Technical Effects

1. The human serum albumin-conjugated drug of the present disclosure can be recognized and internalized by tumor cells, and the therapeutic drug can be accurately delivered into the tumor, thereby enhancing the targeting effect of the drug, and avoiding the indiscriminate killing on normal tissues, organs and cells by the drug.

2. The human serum albumin-conjugated drug of the present disclosure can circulate steadily in the blood. The drug obtained by conjugating human serum albumin with a toxin molecule through a stable linker unit in the blood only breaks apart under acidic condition when it reaches tumor cells and releases the cytotoxin, which addresses the technical problem of off-target toxicity caused by the instability of albumin nano-delivery system in the blood.

3. The conjugation of human serum albumin and the toxin molecule enhances the permeability of the toxin molecule to tumor cells and enhances the therapeutic effect.

4. Human serum albumin replaces antibodies as the carrier of the conjugated drug, which addresses the severe side effects of the antibody-drug conjugate caused due to the immunogenicity of the antibodies.

5. The half-life of human serum albumin is 21 days, reducing the number of medications for patients and alleviating their pain.

### BRIEF DESCRIPTION OF THE DRAWINGS

FIG. 1 shows the result of the treatment of data from flow cytometry experiment to study the penetration of the multifunctional nanostructure into MCF-7 cells. FIG. 1a shows the effect of the penetration of HSA-Cy5-HcyTFAc-MMAE into MCF-7 cells. FIG. 1b shows the effect of the penetration of HSA-Cy5-HcyTFAc-MMAF into MCF-7 cells. FIG. 1c shows the effect of the penetration of the control drug into MCF-7 cells. It can be seen from the provided data that the penetration rate of the albumin conjugate reaches 80% after incubation for 4 hours.

FIG. 2 shows the apoptosis assay of A-549 cells by flow cytometry. The apoptosis efficiency of untreated cells (FIG. 2a) is 3.8%; the apoptosis efficiency of MMAF-treated cells (FIG. 2b) is 12.3%; and the apoptosis efficiency of HSA-Cy5-HcyTFAc-MMAF-treated cells (FIG. 2c) is 33.5%. Compared with MMAF-treated cells, the apoptosis rate of HSA-Cy5-HcyTFAc-MMAF-treated cells is significantly higher, indicating that the conjugated drug penetrates into the cells better.

### DESCRIPTION OF THE EMBODIMENTS

In order to make the technical solutions and beneficial effects of the present disclosure more apparent and easier to understand, the following detailed description is provided through specific embodiments. The accompanying drawings are not necessarily drawn to scale, and localized features may be enlarged or reduced to show details of localized features more clearly. Unless otherwise defined, the technical and scientific terms used herein have the same meanings as those in the technical field to which the present disclosure belongs.

In one aspect, the present disclosure provides an albumin conjugate, or a pharmaceutically acceptable salt or a solvate thereof, where the albumin conjugate includes albumin and HcyTFAc,
the albumin is selected from a group consisting of:
a) a protein including an amino acid sequence as set forth in SEQ ID NO:1, in some implementations, a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1;
the HcyTFAc has a structure of

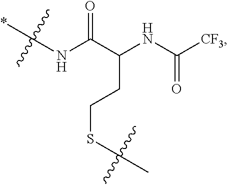

where * is a site directly linked to the albumin.

SEQ ID NO: 1
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL

In some implementations, the albumin conjugate includes a toxin molecule.

In some implementations, the toxin molecule is selected from a group consisting of an antitubulin agent, maytansine, paclitaxel, camptothecin, duocarmycin, pyrrolobenzodiazepine (PBD), Eribulin, adriamycin, doxorubicin, gemcitabine, amethopterin, fluorouracil, Irinotecan, taxol, bleomycin, mitomycin, cytarabine, ramycin, vinblastine, vincristine, morpholine-adriamycin, trifluorothymidine, Dxd, a nucleic acid, a nuclide, and a metal.

In some implementations, the toxin molecule is selected from a group consisting of the antitubulin agent, the maytansine, the paclitaxel, the camptothecin, the duocarmycin, the pyrrolobenzodiazepine (PBD), the Eribulin, the adriamycin, the Irinotecan, the Dxd, the nucleic acid, the nuclide, and the metal.

In some implementations, the antitubulin agent is selected from a group consisting of a tubulin inhibitor.

In some implementations, the antitubulin agent is selected from auristatin.

In some implementations, the antitubulin agent is selected from auristatin E, auristatin F, and auristatin D.

In some implementations, the antitubulin agent is selected from monomethyl auristatin E (MMAE), monomethyl auristatin F (MMAF), and monomethyl auristatin D (MMAD).

In some implementations, the albumin conjugate includes a linker.

In some implementations, the linker is selected from a group consisting of a thioether bond, a disulfide bond, an aminoamide bond, an amide bond, a peptide bond, and

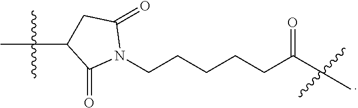 [SMILES: *C(=O)CCCCCN1C(=O)CC(*)C1=O]

In some implementations, the linker is selected from a group consisting of the disulfide bond, the aminoamide bond, the amide bond, and the

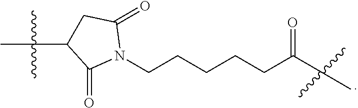 [SMILES: *C(=O)CCCCCN1C(=O)CC(*)C1=O]

In some implementations, the linker is selected from the

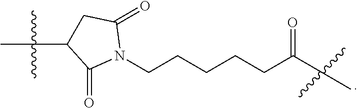 [SMILES: *C(=O)CCCCCN1C(=O)CC(*)C1=O]

In some implementations, the albumin conjugate includes a linker unit.

In some implementations, the linker unit is selected from a group consisting of an enzyme-cleavable linker unit and an enzyme-uncleavable linker unit.

In some implementations, the enzyme-cleavable linker unit is selected from a group consisting of a protease cleavable linker unit.

Preferably, the protease cleavable linker unit includes a combination of two or more selected from a group consisting of valine, citrulline, alanine, glycine, lysine, phenylalanine, glutamic acid, serine, and aspartic acid.

In some implementations, the protease cleavable linker unit includes a combination of one or more selected from a group consisting of valine-citrullinedipeptide, valine-alaninedipeptide, and glycine-glycine-phenylalanine-glycinetetrapeptide.

In some implementations, the protease cleavable linker unit is selected from a group consisting of valine-citrulline-p-aminobenzyloxy (Val-Cit-PAB), valine-alanine-p-aminobenzyloxy (Val-Ala-PAB), glycine-glycine-phenylalanine-glycine (Gly-Gly-Phe-Gly), valine-citrulline-p-aminobenzyloxy (Val-Cit-PAB), valine-alanine-p-aminobenzyloxy (Val-Ala-PAB), and glycine-glycine-phenylalanine-glycine (Gly-Gly-Phe-Gly).

In some implementations, the albumin conjugate has a structure as shown in Formula I:

HSA-(R-linker-linker unit-D)ₙ (Formula I)
where:
HSA is selected from a group consisting of:
a) a protein including an amino acid sequence as set forth in SEQ ID NO:1, in some implementations, a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1;
R is selected from HcyTFAc;
D is selected from a toxin molecule;
where the HcyTFAc has a structure of

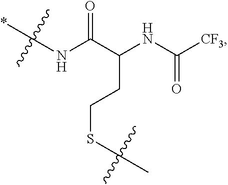

where * is a site directly linked to the albumin;
n is an integer selected from 1-16.

In some implementations, the n is selected from 1, 2, 3, 4, 5, 6, 7, 8, 9, 10, 11, 12, 13, 14, 15, 16.

In some implementations, the n is selected from 2, 3, 4, 5, 6, 7, 8.

In some implementations, the n is selected from 2, 3, 4, 5.

In some implementations, the toxin molecule in Formula I is selected from a group consisting of an antitubulin agent, maytansine, paclitaxel, camptothecin, duocarmycin, pyrrolobenzodiazepine (PBD), Eribulin, adriamycin, doxorubicin, gemcitabine, amethopterin, fluorouracil, taxol, bleomycin, mitomycin, cytarabine, ramycin, vinblastine, vincristine, morpholine-adriamycin, trifluorothymidine, Dxd, a nucleic acid, a nuclide, and a metal.

In some implementations, the toxin molecule is selected from a group consisting of the antitubulin agent, the maytansine, the paclitaxel, the camptothecin, the duocarmycin, the pyrrolobenzodiazepine (PBD), the Eribulin, the adriamycin, the irinotecan, the Dxd, the nucleic acid, the nuclide, and the metal.

In some implementations, the antitubulin agent is selected from a group consisting of a tubulin inhibitor.

In some implementations, the antitubulin agent is selected from auristatin.

In some implementations, the antitubulin agent is selected from auristatin E, auristatin F, and auristatin D.

In some implementations, the antitubulin agent is selected from monomethyl auristatin E (MMAE), monomethyl auristatin F (MMAF), and monomethyl auristatin D (MMAD).

MMAE has a structure of

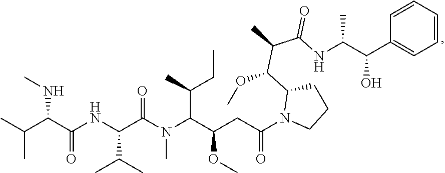 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

### and its CAS RN is 474645-27-7.

MMAF has a structure of

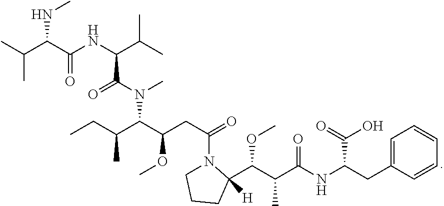 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

### and its CAS RN is 745017-94-1.

MMAD has a structure of

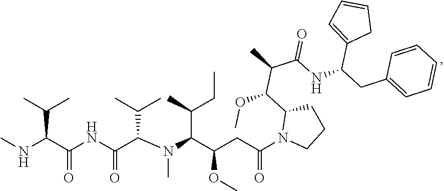 [SMILES: CC[C@H](C)[C@@H]([C@@H](CC(=O)N1CCC[C@H]1[C@H](OC)[C@@H](C)C(=O)N[C@@H](Cc1ccccc1)C1=CC=CC1)OC)N(C)[C@H](C(=O)NC(=O)[C@@H](NC)C(C)C)C(C)C]

### and its CAS RN is 203849-91-6.

n some implementations, the toxin molecule is selected from MMAF-OMe.

MMAF-OMe has a structure of

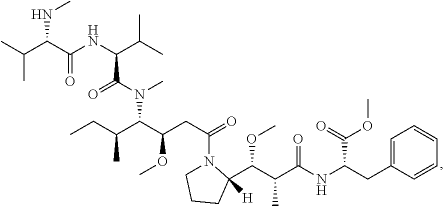 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@@H](NC)C(C)C)C(C)C]

### and its CAS RN is 863971-12-4.

In some implementations, the toxin molecule is selected from Dxd.

Dxd has a structure of

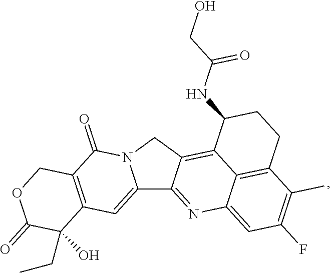 [SMILES: CC[C@@]1(O)C(=O)OCc2c1cc1n(c2=O)Cc2c-1nc1cc(F)c(C)c3c1c2[C@@H](NC(=O)CO)CC3]

### and its CAS RN is 1599440-33-1.

In some implementations, the toxin molecule is selected from SN-38.

SN-38 has a structure of

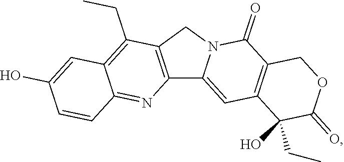 [SMILES: CCc1c2c(nc3ccc(O)cc13)-c1cc3c(c(=O)n1C2)COC(=O)[C@]3(O)CC]

### and its CAS RN is 86639-52-3.

In some implementations, the toxin molecule is selected from DM1.

DM1 has a structure of

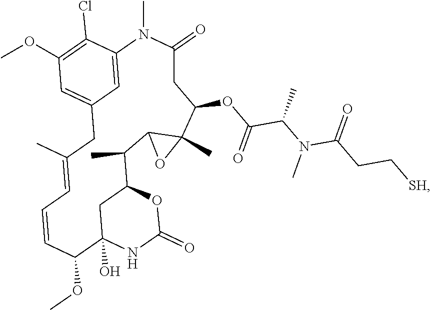 [SMILES: COc1cc2cc(c1Cl)N(C)C(=O)C[C@@H](OC(=O)[C@H](C)N(C)C(=O)CCS)[C@]1(C)OC1[C@H](C)[C@@H]1C[C@@](O)(NC(=O)O1)[C@H](OC)/C=C\C=C(/C)C2]

### and its CAS RN is 139504-50-0.

In some implementations, the toxin molecule is selected from

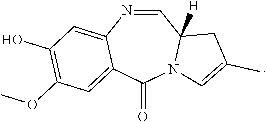

In some implementations, the toxin molecule is selected from

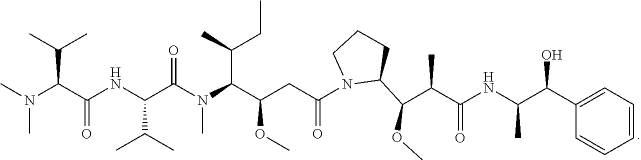 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C)C(C)C]

In some implementations, the toxin molecule is selected from

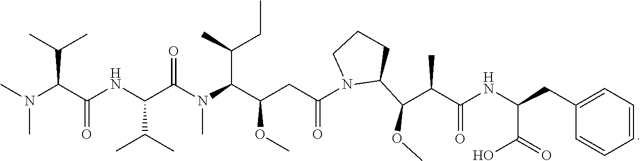 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C)C(C)C]

In some implementations, the albumin conjugate includes a linker.

In some implementations, the linker is selected from a group consisting of a thioether bond, a disulfide bond, an aminoamide bond, an amide bond, a peptide bond, and

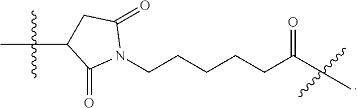 [SMILES: *C(=O)CCCCCN1C(=O)CC(*)C1=O]

In some implementations, the linker is selected from a group consisting of the disulfide bond, the aminoamide bond, the amide bond, and the

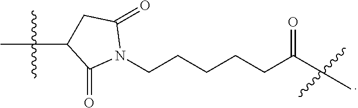 [SMILES: *C(=O)CCCCCN1C(=O)CC(*)C1=O]

In some implementations, the linker is selected from the

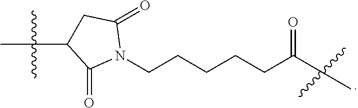 [SMILES: *C(=O)CCCCCN1C(=O)CC(*)C1=O]

In some implementations, the linker unit is selected from a group consisting of an enzyme-cleavable linker unit and an enzyme-uncleavable linker unit.

In some implementations, the enzyme-cleavable linker unit is selected from a group consisting of a protease cleavable linker unit.

In some implementations, the protease cleavable linker unit includes a combination of two or more selected from a group consisting of valine, citrulline, alanine, glycine, lysine, phenylalanine, glutamic acid, serine, and aspartic acid.

In some implementations, the protease cleavable linker unit includes a combination of one or more selected from a group consisting of valine-citrullinedipeptide, valine-alaninedipeptide, and glycine-glycine-phenylalanine-glycinetetrapeptide.

In some implementations, the protease cleavable linker unit is selected from a group consisting of valine-citrulline-p-aminobenzyloxy (Val-Cit-PAB), valine-alanine-p-aminobenzyloxy (Val-Ala-PAB), glycine-glycine-phenylalanine-glycine (Gly-Gly-Phe-Gly), valine-citrulline-p-aminobenzyloxy (Val-Cit-PAB), valine-alanine-p-aminobenzyloxy (Val-Ala-PAB), and glycine-glycine-phenylalanine-glycine (Gly-Gly-Phe-Gly).

In some implementations, the albumin conjugate is selected from a group consisting of conjugates below:

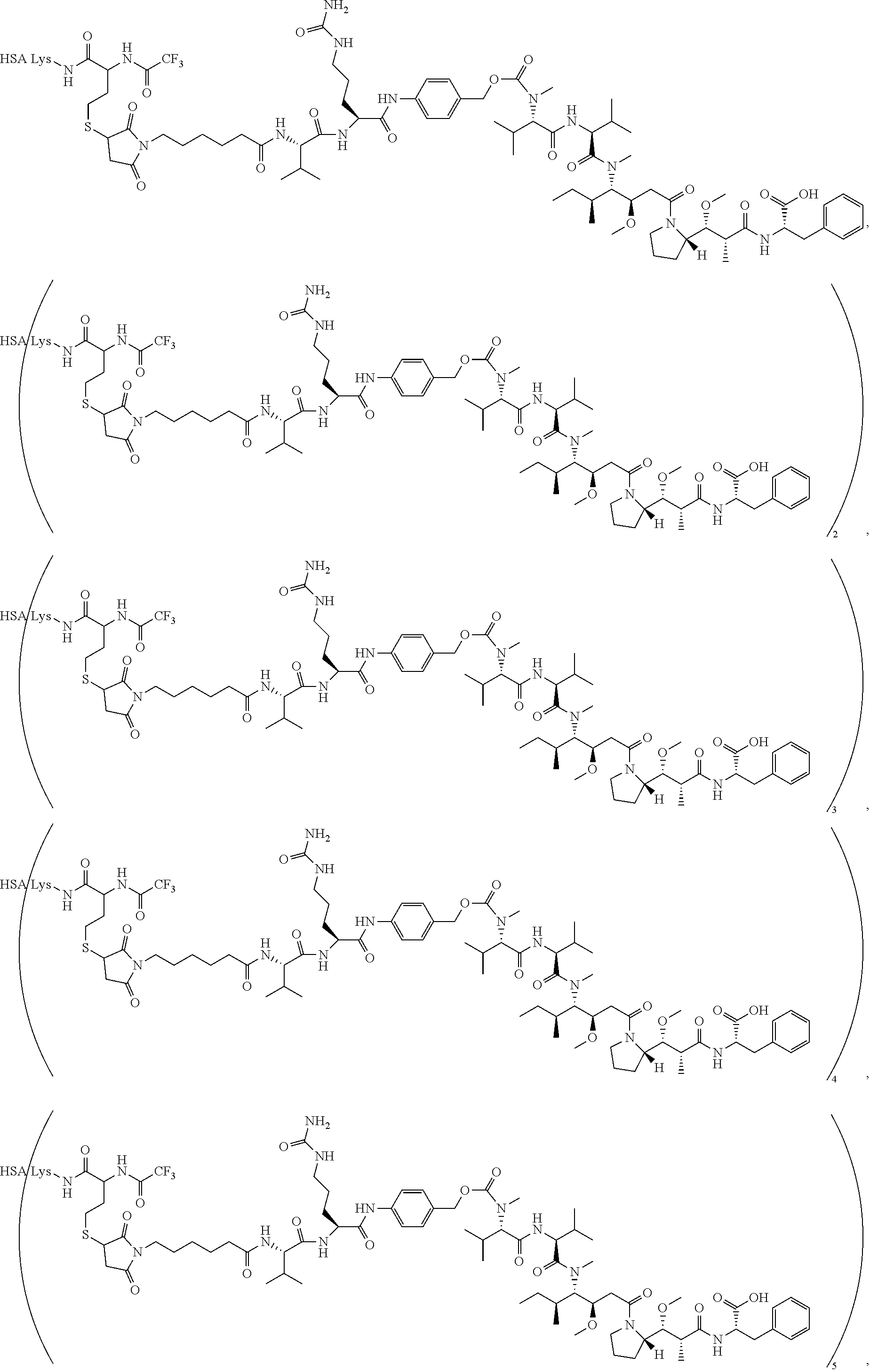

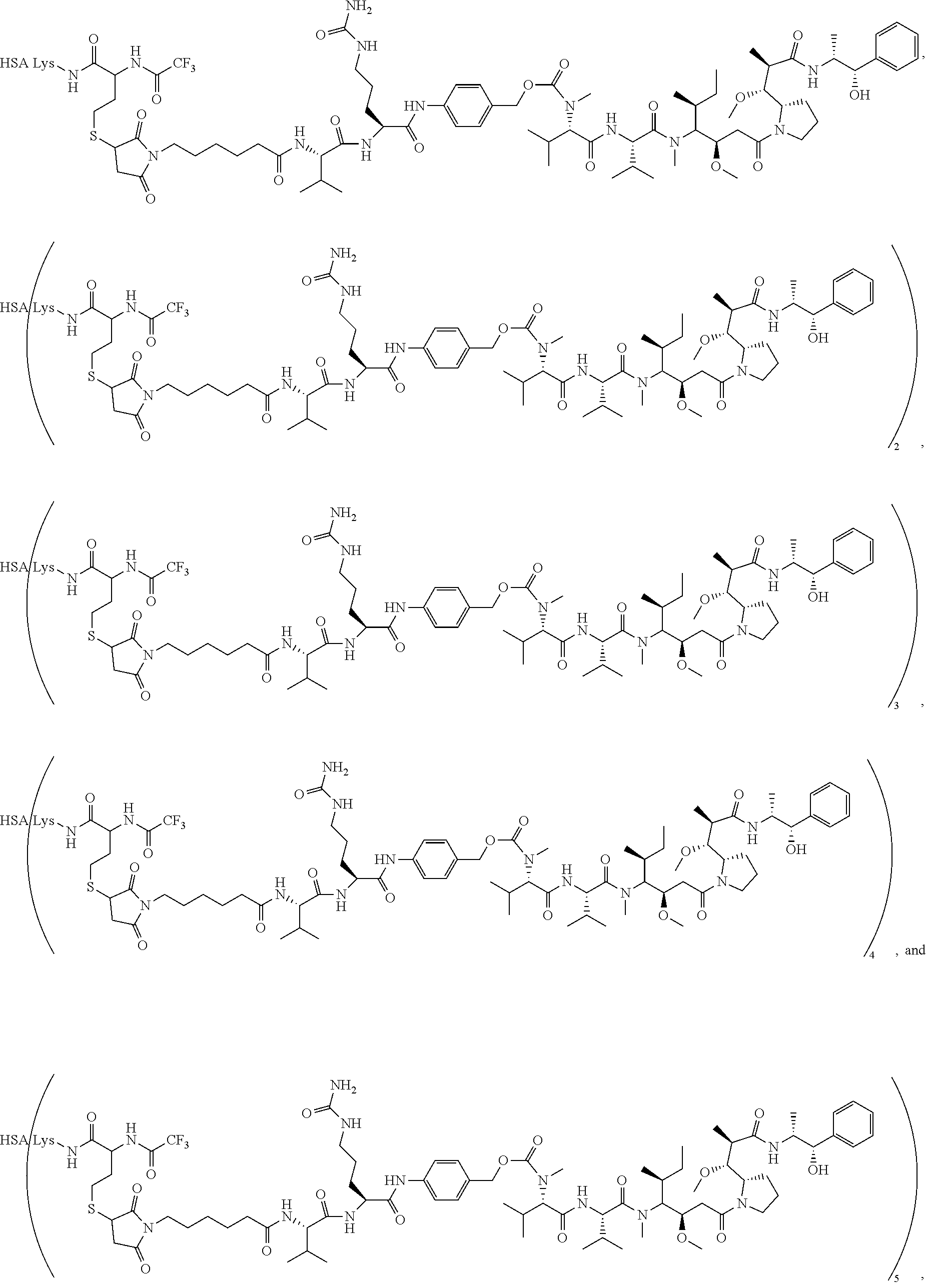

where Lys is a lysine residue in the amino acid sequence of the albumin.

The present disclosure provides a method for preparing the albumin conjugate, specifically as below:
S1: reacting an albumin with HTLTFAc

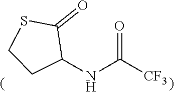 [SMILES: O=C1SCCC1NC(=O)C(F)(F)F]

to obtain an albumin-HcyTFAc;
S2: reacting the albumin-HcyTFAc prepared from S1 with a linker-linker unit-toxin molecule to obtain the albumin conjugate.

The present disclosure provides a pharmaceutical composition including a therapeutically effective amount of the albumin conjugate, or the pharmaceutically acceptable salt or the solvate thereof, and one or more pharmaceutically acceptable carriers, diluents or excipients.

In some implementations, the unit dose of the pharmaceutical composition is 0.001 mg-1000 mg.

In some implementations, based on the total weight of the composition, the pharmaceutical composition includes 0.01%-99.99% of the conjugated compound. In some implementations, the pharmaceutical composition includes 0.1%-99.9% of the conjugated compound. In some implementations, the pharmaceutical composition includes 0.5%-99.5% of the conjugated compound. In some implementations, the pharmaceutical composition includes 1%-99% of the conjugated compound. In some implementations, the pharmaceutical composition includes 2%-98% of the conjugated compound. In some implementations, the pharmaceutical composition includes 5%-95% of the conjugated compound.

In some implementations, based on the total weight of the composition, the pharmaceutical composition includes 0.01%-99.99% of the pharmaceutically acceptable carriers, diluents or excipients. In some implementations, the pharmaceutical composition includes 0.1%-99.9% of the pharmaceutically acceptable carriers, diluents or excipients. In some implementations, the pharmaceutical composition includes 0.5%-99.5% of the pharmaceutically acceptable carriers, diluents or excipients. In some implementations, the pharmaceutical composition includes 1%-99% of the pharmaceutically acceptable carriers, diluents or excipients. In some implementations, the pharmaceutical composition includes 2%-98% of the pharmaceutically acceptable carriers, diluents or excipients.

All conjugated compounds involved in the present disclosure and mixtures and compositions including the conjugated compound of the present disclosure can be given into an organism through any route of administration. The route of administration may be oral administration, intravenous injection, subcutaneous injection, intratumor injection, rectal administration, vaginal administration, sublingual administration, nasal inhalation, oral inhalation, eye dropping, or may be local or systemic percutaneous administration.

All conjugated compounds involved in the present disclosure and mixtures and compositions including the conjugated compound of the present disclosure can be formulated as a single dosage form containing the active conjugated compound of the present disclosure and carriers, excipients, and the like, and the dosage form may be tablets, capsules, injection, granules, powder, suppositories, pills, cream, paste, gel, dust, oral solution, inhalers, suspension, dry suspension, patches, lotion, etc. These dosage forms may contain ingredients commonly used in pharmaceutical preparations, such as a diluent, an absorbent, a wetting agent, a binder, a disintegrant, a colorant, a pH regulator, an antioxidant, an antibacterial agent, an iso-osmotic adjusting agent, an anti-adherent, etc.

Suitable formulations for each of these dosage forms are publicly available, for example, Remington: The Science and Practice of Pharmacy, 21st Edition, Lippincott Williams & Wilkins, published in 2006, and Rowe, Raymond C. Handbook of Pharmaceutical Excipients, Chicago, Pharmaceutical Press, published in 2005. Therefore, these dosage forms can be easily prepared by those skilled in the art.

Depending on the nature of the disease suffered by different individuals, the intensity, the age, gender, weight of the patient, the route of administration and other factors, different dosages can be selected for administration. The conjugated compound of the present disclosure can be administered in a daily dosage of 0.01 to 500 mg/kg, preferably in a daily dosage of 1 to 100 mg/kg, which can be administered as a single or multiple doses.

The present disclosure provides use of the albumin conjugate, or the pharmaceutically acceptable salt or the solvate thereof or the composition for preventing and/or treating a cancer.

In some implementations, the cancer is selected from a group consisting of breast cancer, ovarian cancer, cervical cancer, uterine cancer, prostate cancer, renal cancer, urinary system cancer, bladder cancer, liver cancer, gastric cancer, endometrial cancer, salivary gland cancer, esophageal cancer, lung cancer, colon cancer, rectal cancer, colorectal cancer, bone cancer, skin cancer, thyroid cancer, pancreatic cancer, brain tumor, melanoma, neuroglioma, neuroblastoma, glioblastoma multiforme, sarcoma, lymphoma, and leukemia.

In some implementations, the cancer is selected from a group consisting of breast cancer, ovarian cancer, cervical cancer, uterine cancer, prostate cancer, renal cancer, urinary system cancer, bladder cancer, liver cancer, gastric cancer, esophageal cancer, lung cancer, colorectal cancer, brain tumor, and melanoma.

The present disclosure provides use of the albumin conjugate, or the pharmaceutically acceptable salt or the solvate thereof or the composition in preparation of a drug for preventing and/or treating a cancer.

In some implementations, the cancer is selected from a group consisting of breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, endometrial cancer, salivary gland cancer, esophageal cancer, lung cancer, colon cancer, rectal cancer, colorectal cancer, bone cancer, skin cancer, thyroid cancer, pancreatic cancer, brain tumor, melanoma, neuroglioma, neuroblastoma, glioblastoma multiforme, sarcoma, and leukemia.

In some implementations, the cancer is selected from a group consisting of breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, esophageal cancer, lung cancer, colorectal cancer, brain tumor, and melanoma.

The present disclosure provides the albumin conjugate, or the pharmaceutically acceptable salt or the solvate thereof or the composition, which is used for preventing and/or treating a cancer.

In some implementations, the cancer is selected from a group consisting of breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, endometrial cancer, salivary gland cancer, esophageal cancer, lung cancer, colon cancer, rectal cancer, colorectal cancer, bone cancer, skin cancer, thyroid cancer, pancreatic cancer, brain tumor, melanoma, neuroglioma, neuroblastoma, glioblastoma multiforme, sarcoma, and leukemia.

In some implementations, the cancer is selected from a group consisting of breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, esophageal cancer, lung cancer, colorectal cancer, brain tumor, and melanoma.

The present disclosure provides a method for preventing and/or treating a cancer in a subject in need thereof, including:

administering to the subject a therapeutically-effective amount of the albumin conjugate, or the pharmaceutically acceptable salt or the solvate thereof.

In some implementations, the cancer may include breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, endometrial cancer, salivary gland cancer, esophageal cancer, lung cancer, colon cancer, rectal cancer, colorectal cancer, bone cancer, skin cancer, thyroid cancer, pancreatic cancer, brain tumor, melanoma, neuroglioma, neuroblastoma, glioblastoma multiforme, sarcoma, and leukemia.

In some implementations, the cancer may include breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, esophageal cancer, lung cancer, colorectal cancer, brain tumor, and melanoma

The present disclosure provides a method for preventing and/or treating a cancer in a subject in need thereof, including:

administering to the subject a therapeutically-effective amount of a pharmaceutical composition,

where the pharmaceutical composition comprises a therapeutically effective amount of the albumin conjugate, or the pharmaceutically acceptable salt or the solvate thereof described above, and one or more pharmaceutically acceptable carriers, diluents or excipients.

In some implementations, the cancer may include breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, endometrial cancer, salivary gland cancer, esophageal cancer, lung cancer, colon cancer, rectal cancer, colorectal cancer, bone cancer, skin cancer, thyroid cancer, pancreatic cancer, brain tumor, melanoma, neuroglioma, neuroblastoma, glioblastoma multiforme, sarcoma, and leukemia.

In some implementations, the cancer may include breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, esophageal cancer, lung cancer, colorectal cancer, brain tumor, and melanoma

### Definition of Terms:

Unless otherwise specified, terms used in the description and claims have the following meanings.

As used herein, “albumin conjugate” means that albumin is linked to a biologically active drug through a stable linker unit.

As used herein, “conjugation” refers to covalent, ionic or hydrophobic interaction by which parts of a molecule are bound together and held in close proximity.

The three-letter codes and single-letter codes for amino acids used in the present disclosure are as described in J. biol. Chem, 1968, 243, 3558.

“Albumin” as used herein refers to a) a protein including an amino acid sequence as set forth in SEQ ID NO: 1, and in some implementations, a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1.

In some implementations, c) a protein having 80%, 81%, 82%, 83%, 84%, 85%, 86%, 87%, 88%, 89%, 90%, 91%, 92%, 93%, 94%, 95%, 96%, 97%, 98%, 99% or 100% sequence identity compared with SEQ ID NO:1.

The term “conservative substitution” refers to an amino acid substitution that does not adversely affect or change the biological activity of the protein/polypetide including the amino acid sequence. For example, conservative substitutions can be introduced by standard technologies known in the art, such as site-directed mutagenesis or PCR-mediated mutagenesis. Conservative amino acid substitutions include substitutions in which an amino acid residue is replaced with an amino acid residue having a similar side chain, such as replacement with a residue that is physically or functionally similar to the corresponding amino acid residue (e.g., having similar size, shape, charge, chemical properties, including the ability to form covalent bonds or hydrogen bonds, etc.). Families of amino acid residues having similar side chains have been defined in the art. These families include amino acids having basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Therefore, it is preferable to replace a corresponding amino acid residue with another amino acid residue from the family having the same side chain. Methods for identifying conservative substitutions of amino acids are well known in the art (see, e.g., Brummell et, al., Biochem. 32:1180-1187 (1993); Kobayashi et, al., Protein Eng.12 (10): 879-884 (1999); and Burks et, al., Proc. Natl Acad. Set USA 94:412-417 (1997), all incorporated herein by reference).

The term “identity” is used to refer to the match in sequences between two polypeptides or two nucleic acids. When a position in two sequences being compared is occupied by the same base or amino acid monomer subunit (e.g., a position in each of two DNA molecules is occupied by adenine, or a position in each of two polypeptides is occupied by lysine), then the respective molecules are identical at that position. The “percent identity” between two sequences is a function of the number of matching positions shared by the two sequences divided by the number of positions being compared x 100. For example, if 6 out of 10 positions in two sequences match, the two sequences are 60% identical. For example, DNA sequences CTGACT and CAGGTT share an identity of 50% (3 out of a total of 6 positions match). In general, two sequences are aligned to produce maximum identity for comparison. Such alignment can be achieved by performing the method of Needleman et, al. (1970) J. Mol. Biol. 48:443-453 conveniently using a computer program such as Align program (DNAstar, Inc.). The percent identity between two amino acid sequences can also be determined using an algorithm of E. Meyers and W. Miller (Comput. Appl Biosci., 4:11-17 (1988)) that has been integrated into the ALIGN program (version 2.0), using PAM120 weight residue table, with a gap length penalty of 12 and a gap penalty of 4. Additionally, the percent identity between two amino acid sequences can also be determined using an algorithm of Needleman and Wunsch (J Mol Biol. 48:444-453 (1970)) that has been integrated into the GAP program of the GCG software package (available from www.gcg.com), using a Blossum 62 matrix or a PAM250 matrix, with a gap weight of 16, 14, 12, 10, 8, 6 or 4 and a length weight of 1, 2, 3, 4, 5 or 6.

The term “toxin molecule” refers to a cytotoxic drug, represented as D, which is a chemical molecule capable of exerting a strong effect in tumor cells to disrupt their normal growth. The cytotoxic drug can kill tumor cells at sufficiently high concentration in principle. However, due to the lack of specificity, they may cause apoptosis of normal cells while killing tumor cells, thus leading to serious side effects. This term includes a toxin, such as a bacterium, fungus, plant-or animal-derived micromolecular toxin or enzyme-active toxin, radioactive isotope, toxic drug, chemotherapeutic drug, antibiotic, and nuclease, preferably toxic drug.

The term “toxic drug” refers to a substance that inhibits or prevents the function of cells or causes cells death or disruption, including a toxin and other compounds suitable for tumor treatment.

As used herein, “mitomycin” refers to members of drug families containing aziridine isolated from Streptomyces caespitosus or Streptomyces lavendulae, particularly including mitomycin C and mitomycin A.

As used herein, “adriamycin” refers to members of anthracycline family derived from Streptomycespeucetius var. caesius, including doxorubicin, daunorubicin, epirubicin, and idamycin.

As used herein, “camptothecin” refers to members of alkaloid family isolated from Camptotheca acuminata and chemical derivatives thereof, including camptothecin, Irinotecan, Topotecan, and Rubitecan.

The term “linker fragment” or “linker unit” refers to a chemical structural fragment or bond with one end linked to a ligand and the other end linked to a drug, or linked to other linkers before being linked to the drug.

The term “linker” refers to a chemical structural fragment or bond with one end linked to a ligand and the other end linked to the “linker fragment” or “linker unit”.

The “linker” or “linker fragment” or “linker unit” as used herein is a divalent group, which is a moiety obtained by removing two H on the basis of the compound, for example:
Val-Cit-PAB has a structure of

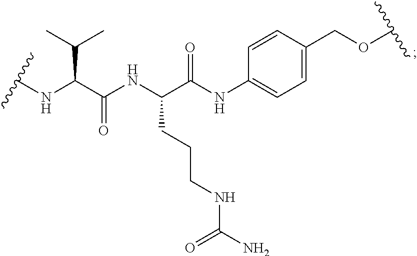 [SMILES: *N[C@H](C(=O)N[C@@H](CCCNC(N)=O)C(=O)Nc1ccc(CO*)cc1)C(C)C]

Val-Ala-PAB has a structure of

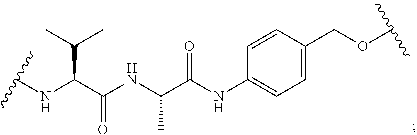 [SMILES: *N[C@H](C(=O)N[C@@H](C)C(=O)Nc1ccc(CO*)cc1)C(C)C]

Gly-Gly-Phe-Gly has a structure of

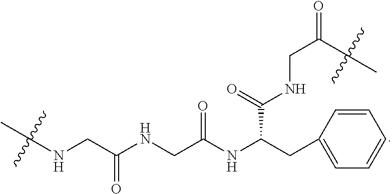 [SMILES: *NCC(=O)NCC(=O)N[C@@H](Cc1ccccc1)C(=O)NCC(*)=O]

The term “about” should be understood by those skilled in the art and will vary somewhat with the context in which it is used. If the meaning is unclear to those skilled in the art from the context in which the term is used, the term “about” means that the deviation does not exceed plus or minus 10% of the particular value or range stated.

As used herein, the term “pharmaceutical composition” represents a mixture containing one or more of the conjugated compounds described herein or physiological/pharmaceutical salts or prodrugs thereof and other chemical components, and the other components are physiological/pharmaceutical carriers and excipients for example. The pharmaceutical composition aims to promote the administration to organisms, so as to facilitate the absorption of active ingredients and thus exert the biological activity.

As used herein, the term “salt” refers to a salt of the conjugated compound of the present disclosure, which is safe, effective and has corresponding biological activity in mammals. The salt can be individually prepared during the final separation and purification of the conjugated compound, or through the reaction of a suitable group with a suitable base or acid. The base commonly used to form the pharmaceutically acceptable salt includes an inorganic base and an organic base. The acid commonly used to form the pharmaceutically acceptable salt includes an inorganic acid and an organic acid.

With respect to a drug or a pharmacological active agent, the term “therapeutically effective amount” refers to an amount of a drug or an agent that is nontoxic but sufficient to achieve an expected effect. The determination of the effective amount varies from person to person, depending on the age and general condition of the recipient and also depending on the specific active substance. The appropriate effective amount in each case can be determined by a person skilled in the art based on routine experiments.

As used herein, the term “solvate” refers to a physical binding of the conjugated compound of the present disclosure to one or more, preferably 1-3, solvent molecules, no matter organic or inorganic solvent molecules. Such physical binding includes a hydrogen bond. In some instances, for example, when one or more, preferably 1-3, solvent molecules are incorporated into the lattice of a crystalline solid, the solvate will be separated. Exemplary solvates include, but not limited to, hydrates, ethanolates, methanolates, and isopropanolates. Solvation processes are well known in the art.

As used herein, the term “pharmaceutically acceptable” means that these conjugated compounds, materials, compositions, and/or dosage forms are, within a scope of reasonable medical judgment, suitable for contact with patient tissues without excessive toxicity, irritation, allergic reaction, or other problems or complications, have a reasonable benefit/risk ratio, and are effective for the intended use.

As used herein, the singular forms of “one”, “a”, and “the” include plural references, and vice versa, unless the context explicitly specifies otherwise.

The terms involved in the present disclosure have been defined above, which can also be understood by persons skilled in the art in conjunction with the prior art. The following is a further description based on the content of the present disclosure and the definitions of the terms.

The preparation of the conjugated compound of the present disclosure and pharmaceutically acceptable salt thereof is further described below in connection with embodiments that are not intended to limit the scope of the present disclosure.

Experimental methods in examples of the present disclosure in which specific conditions are not indicated are usually in accordance with conventional conditions, or in accordance with conditions recommended by the manufacturer of the raw material or commodity. Reagents of no specific origin are conventional reagents commercially purchased in the market.

### Embodiment 1: Preparation of HSA-Cy5-HcyTFAc-MMAE

Cy5

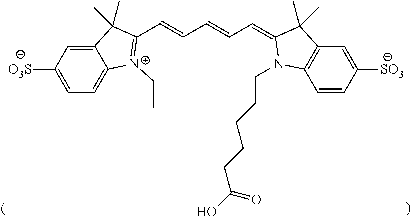

has a maximum absorption at 646 nm, an extinction coefficient of 271000 1/mol cm, and fluoresces at 662 nm. It is capable of tracking the distribution of albumin conjugates within tumor cells by spectrophotometry. Therefore, an protein was firstly modified with a maleimide derivative of Cy5. A method was specifically as below: a solution (110 mg) of HSA in PBS buffer (1.65 mL, 1 mmol, 1.65 μmol) was mixed with Cy5 maleimide (

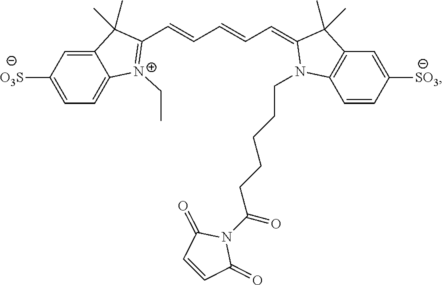

0.67 mg, 34.5 μL, 0.83 μmol) dissolved in DMSO. The reaction was conducted at 40° C. for 24 hours while stirring. The obtained HSA-Cy5 protein conjugate (

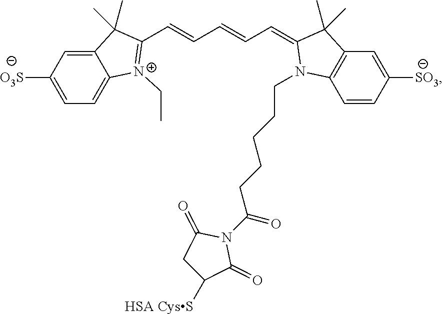

where Cys is the cysteine residue in the amino acid sequence of the albumin) was purified using a filter (Amicon Centriprep 10 YM30, Millipore, Belford) to remove impurities with a molecular weight less than 3000 Da.

The HSA-Cy5 protein conjugate (1510 μL, 0.84 mmol, 1.27 μmol) in PBS was mixed with a 15-fold excess of HTLTFAc

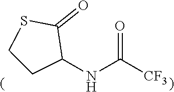 [SMILES: O=C1SCCC1NC(=O)C(F)(F)F]

dissolved in DMSO. The reaction was conducted at 40° C. for 50 the hours while stirring. As described above, obtained HSA-Cy5-HTLTFAc (

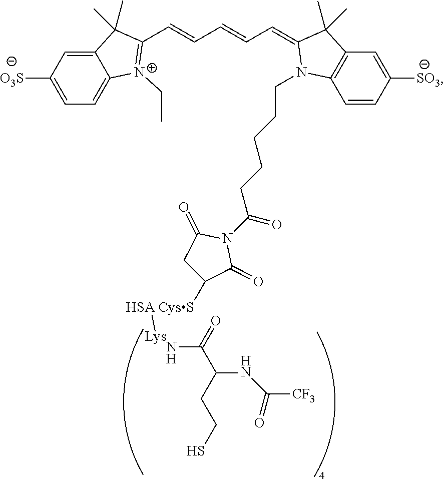

where Lys is the lysine residue in the amino acid sequence of the albumin) protein conjugate was purified using a filter (Amicon Centriprep YM30, Millipore, Belford) to remove impurities with a molecular weight less than 3000 Da.

The HSA-Cy5-HcyTFAc (882 μL, 0.7 mmol, 0.617 μmol) in PBS was mixed with a 6.8-fold excess of MC-Val-Cit-PAB-MMAE

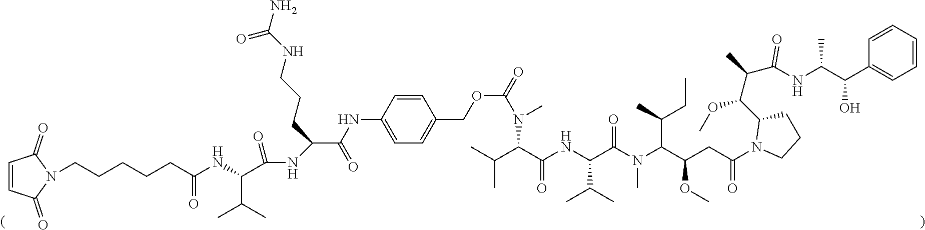 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

dissolved in DMSO. The reaction was conducted at 40° C. for 28 hours while stirring. As described above, the obtained HSA-Cy5-HTLTFAc-MMAE
(

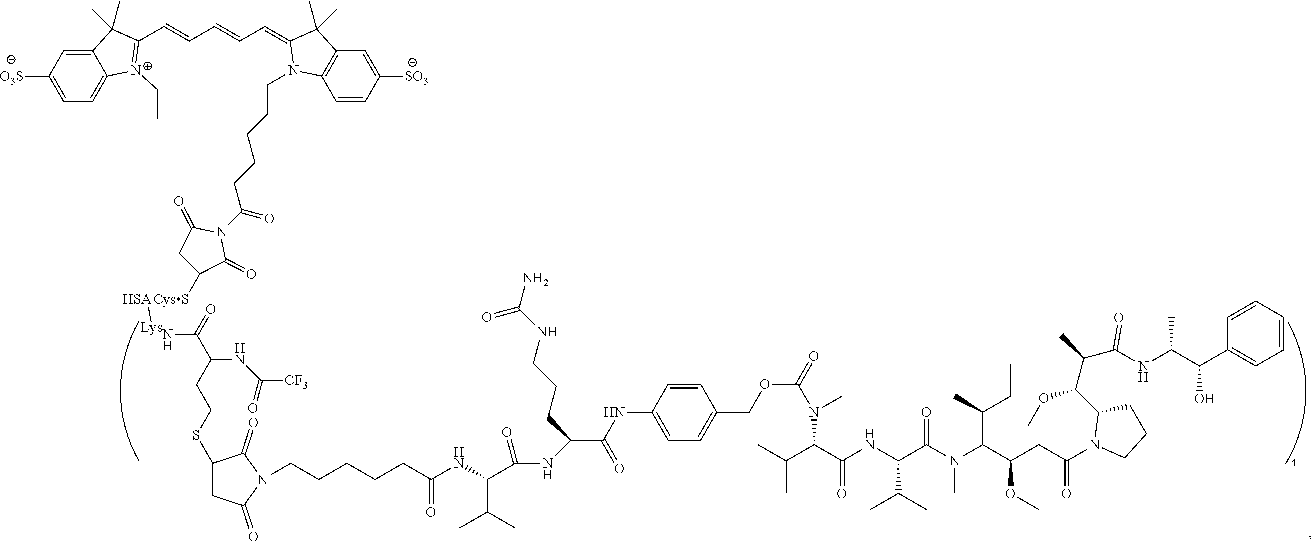

where Cys is the cysteine residue in the amino acid sequence of the albumin, and Lys is the lysine residue in the amino acid sequence of the albumin) protein conjugate was purified using a filter (Amicon Centriprep YM30, Millipore, Belford) to remove impurities with a molecular weight less than 3000 Da.

Absorption spectrum (PBS, pH 7.4): λmax 278 nm (ε=(5.4±0.1)×104), λmax 646 nm (ε=(27.1±0.1)×104). 19F MRI (PBS +D2O, δ, m.d.): 87.75 (c, CF3). MALDI-TOF m/z: calculated MW value for HSA-Cy5-HcyTFAc-MMAE: 68796.6 Da (this includes measured MW value for HSA: 66500 Da, calculated MW value for Cy5 residue: 766 Da, HcyTFAc: 214 Da, MMAE:1316.6 Da). The measured MW value for HSA-Cy5-HcyTFAc was 72730 Da.

### Embodiment 2: Preparation of HSA-Cy5-HcyTFAc-MMAF

A solution (110 mg) of HSA in PBS buffer (1.65 mL, 1 mmol, 1.65 μmol) was mixed with Cy5 maleimide (

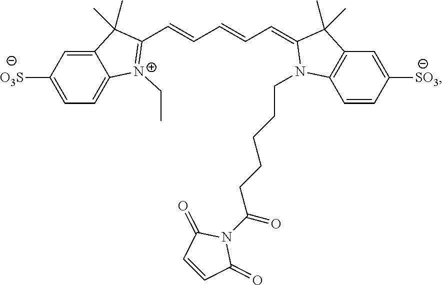

0.67 mg, 34.5 μL, 0.83 μmol) dissolved in DMSO. The reaction was conducted at 40° C. for 24 hours while stirring. The obtained HSA-Cy5 protein conjugate (

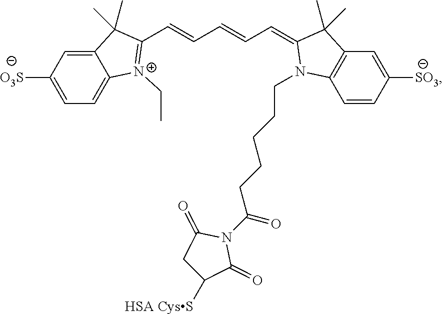

where Cys is the cysteine residue in the amino acid sequence of the albumin) was purified using a filter (Amicon Centriprep YM30, Millipore, Belford) to remove impurities with a molecular weight less than 3000 Da.

The HSA-Cy5 protein conjugate (1510 μL, 0.84 mmol, 1.27 μmol) in PBS was mixed with a 15-fold excess of HTLTFAc

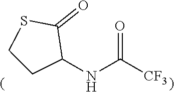 [SMILES: O=C1SCCC1NC(=O)C(F)(F)F]

dissolved in DMSO. The reaction was conducted at 40° C. for 50 hours while stirring. As described above, the obtained HSA-Cy5-HTLTFAc (

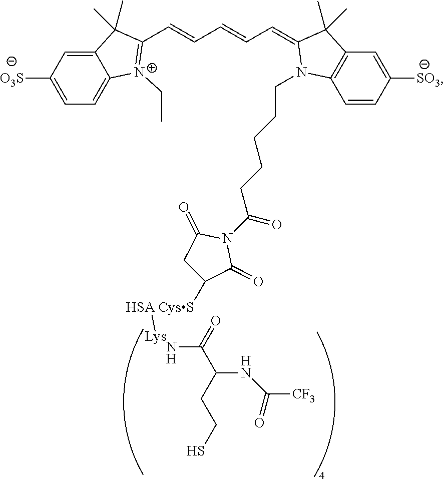

where Cys is the cysteine residue in the amino acid sequence of the albumin, Lys is the lysine residue in the amino acid sequence of the albumin) protein conjugate was purified using a filter (Amicon Centriprep YM30, Millipore, Belford) to remove impurities with a molecular weight less than 3000 Da. Afterwards, the HSA-Cy5-HcyTFAc (882 μL, 0.7 mmol, 0.617 μmol) in PBS was mixed with a 6.8-fold excess of MC-Val-Cit-PAB-MMAF (

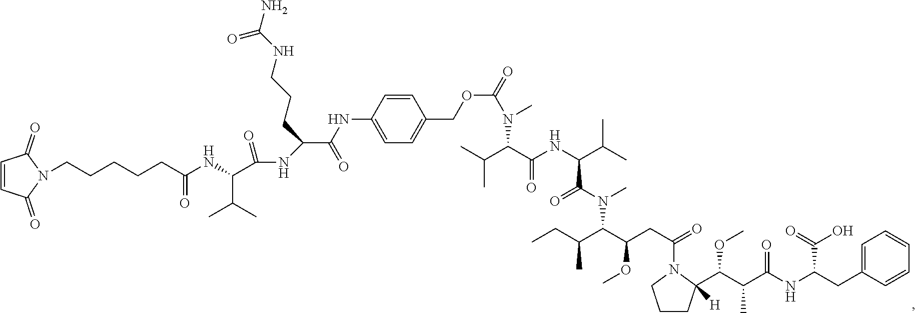 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

5 mg, 176.4 μL, 3.78 μmol) dissolved in DMSO. The ratio of PBS to DMSO in the reaction mixture was 20:1. The reaction was conducted at 40° C. for 28 hours while stirring. As described above, the obtained HSA-Cy5-HTLTFAc-MMAF protein conjugate
(

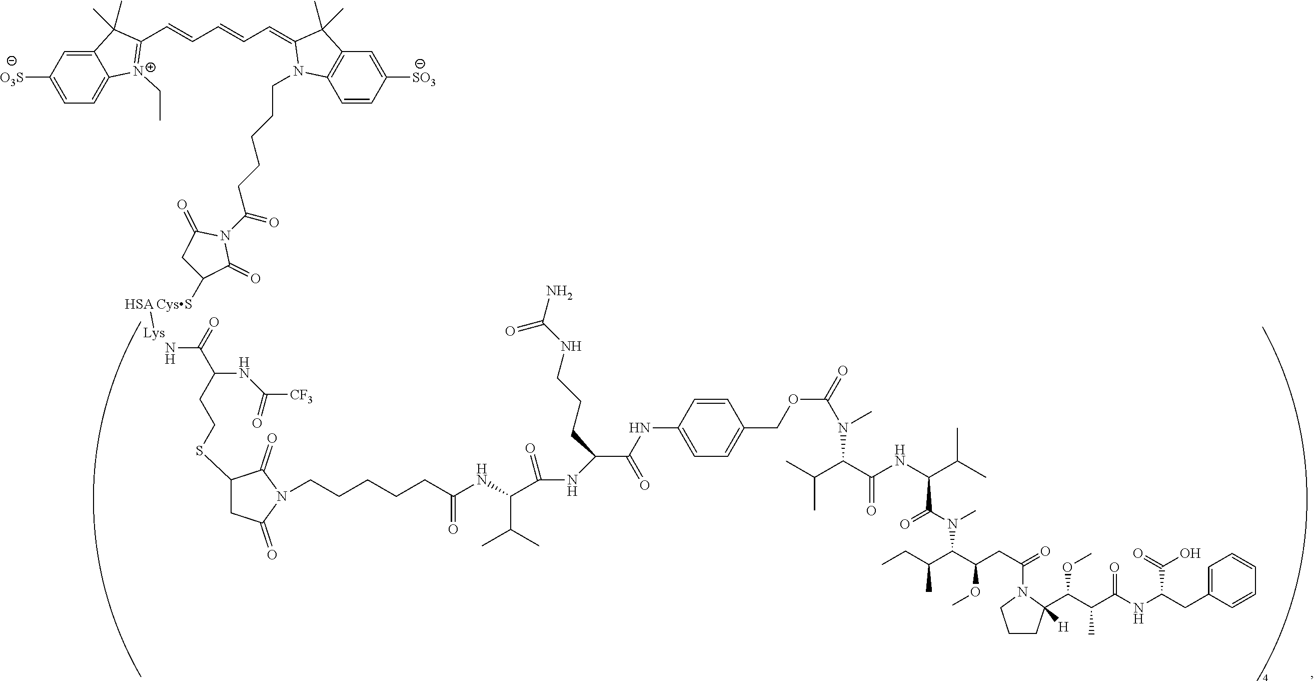

where Cys is the cysteine residue of the amino acid sequence of the albumin, Lys is the lysine residue in the amino acid sequence of the albumin) was purified using a filter (Amicon Centriprep YM30, Millipore, Belford) to remove impurities with a molecular weight less than 3000 Da.

Absorption spectrum (PBS, pH 7.4): λmax 278 nm (ε=(4.2±0.1)×104), λmax 646 nm (ε=(27.1±0.1)×104). Nuclear magnetic resonance 19F (PBS+D20, δ, M.d.):87.80 (s, CF3). MALDI-TOF m/z: calculated MW value for HSA-Cy5-HcyTFAc: 67480 Da (this includes measured MW value for HSA: 66500 Da, calculated MW value for Cy5 residue: 766 Da, HcyTFAc: 214 Da). The measured MW value for HSA-Cy5-HcyTFAc was 68122 Da, which corresponds to one Cy5 residue and four HcyTFAc residues attached to the HSA molecule.

Absorption spectrum (PBS, pH 7.4): λmax 278 nm (ε=(5.2±0.1)×104), λmax 646 nm (ε=(27.1±0.1)×104). Nuclear magnetic resonance 19F (PBS+D20, δ, m.d.):87.70 (s, CF3). MALDI-TOF m/z: calculated MW value for HSA-Cy5-HcyTFAc-MMAF: 68810 Da (this includes measured MW value for HSA: 66500 Da, calculated MW value for Cy5 residue: 766 Da, HcyTFAc: 214 Da, and MMAF: 1330 Da). The measured MW value for HSA-Cy5-HcyTFAc was 71447 Da.

### Activity Example 1

Flow cytometry is a convenient method for assessing the efficiency of penetration of a therapeutic agent into cells, provided that the therapeutic agent contains a fluorescent marker. The essence of the method is to detect the light scattering of cells as they pass through a laser beam in a liquid jet, and the degree of light scattering can give insight into the size and structure of the cells. In addition, the assay also takes into account the fluorescence levels of substances that constitute the cells (autofluorescence) or are introduced into the sample prior to flow cytometry.

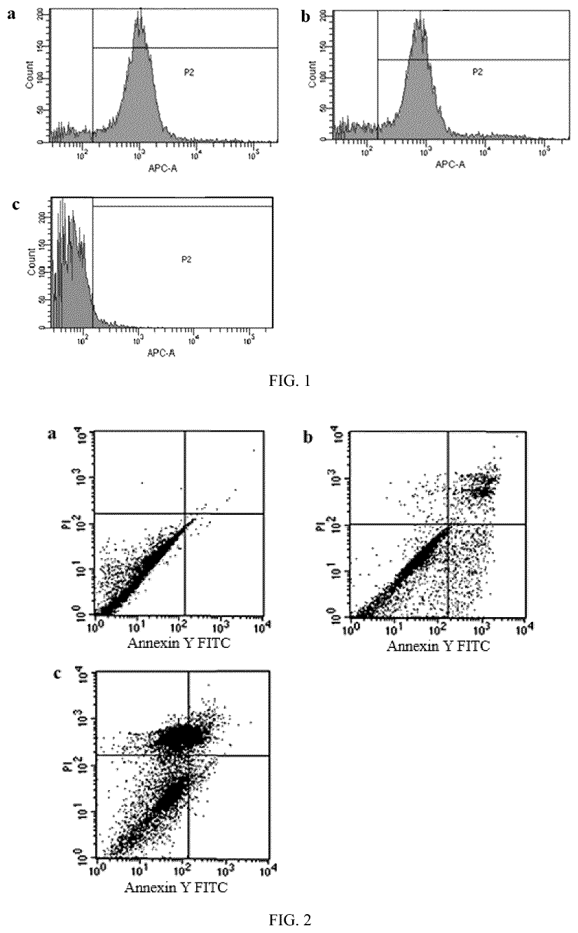

The experiment was conducted using a 6-well microplate. 2×105 MCF-7 cells were placed in 100 μL of complete nutrient medium in each well, then 400 μl of PBS buffer (pH 7.4) containing sample No. 2 or 3 was added into each well. One hour after the addition of the albumin conjugate, the cells were centrifuged (1000 rpm×5 min), washed with PBS buffer for three times, and suspended in 0.5 ml of PBS. The obtained samples were analyzed by flow cytometry using FACSCanto II flow cytometer (Becton Dickinson), and the HSA-Cy5-HcyTFAc-MMAE and HSA-Cy5-HcyTFAc-MMAF samples were analyzed with FACSCanto II flow cytometer (Becton Dickinson) by using FACSDiva program (BD Biosciences) (FIG. 1). The result of FACS analysis shows the efficiency of penetration of the albumin conjugate into MCF-7 cells.

### Activity Example 2

### Study of In Vitro Cytotoxic Activity of Albumin Conjugate

The apoptosis of A-549 cells was analyzed by flow cytometry (FIG. 2). The apoptosis efficiencies of control cells (untreated cells and cells treated with intermediate conjugate HSA-Cy5-HcyTFAc not containing MMAF) were almost the same, 3.8% and 4.3% respectively. In the groups of cells treated with MMAF and HSA-Cy5-HcyTFAc-MMAF respectively, the apoptosis efficiencies were 12.3% and 33.5%, respectively. Therefore, compared with cells treated with MMAF, the apoptosis rate of cells treated with HSA-Cy5-HcyTFAc-MMAF was obviously higher, which was attributed to better penetration of the albumin conjugate into the cells compared to the original toxin.

It should be understood that the above embodiments are exemplary and are not intended to encompass all possible implementations included in the claims. Various modifications and changes can also be made on the basis of the above embodiments without departing from the scope of the present disclosure. Likewise, the various technical features of the above embodiments may be arbitrarily combined to form additional embodiments of the present disclosure that may not have been explicitly described. Therefore, the above embodiments only illustrate several implementations of the present disclosure and do not limit the scope of protection of the present disclosure.

1. An albumin conjugate, or a pharmaceutically acceptable salt or a solvate thereof, wherein the albumin conjugate comprises albumin and HcyTFAc,
the albumin is selected from a group consisting of:
a) a protein comprising an amino acid sequence as set forth in SEQ ID NO:1, preferably a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1;
the HcyTFAc has a structure of 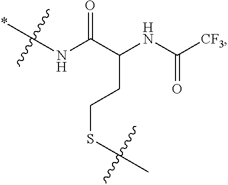
wherein * is a site directly linked to the albumin.

2. The albumin conjugate, or the pharmaceutically acceptable salt or the solvate thereof according to claim 1, wherein the albumin conjugate comprises a toxin molecule;
preferably, the toxin molecule is selected from a group consisting of an antitubulin agent, maytansine, paclitaxel, camptothecin, duocarmycin, pyrrolobenzodiazepine (PBD), Eribulin, adriamycin, doxorubicin, gemcitabine, amethopterin, fluorouracil, Irinotecan, taxol, bleomycin, mitomycin, cytarabine, ramycin, vinblastine, vincristine, morpholine-adriamycin, trifluorothymidine, Dxd, a nucleic acid, a nuclide, and a metal;
preferably, the toxin molecule is selected from a group consisting of the antitubulin agent, the maytansine, the paclitaxel, the camptothecin, the duocarmycin, the pyrrolobenzodiazepine (PBD), the Eribulin, the adriamycin, the Irinotecan, the Dxd, the nucleic acid, the nuclide, and the metal;
preferably, the antitubulin agent is selected from a group consisting of a tubulin inhibitor, preferably selected from auristatin, more preferably selected from auristatin E, auristatin F, and auristatin D, most preferably selected from monomethyl auristatin E (MMAE), monomethyl auristatin F (MMAF), and monomethyl auristatin D (MMAD).

3. The albumin conjugate, or the pharmaceutically acceptable salt or the solvate thereof according to claim 1 or 2, wherein the albumin conjugate comprises a linker;
preferably, the linker is selected from a group consisting of a thioether bond, a disulfide bond, an aminoamide bond, an amide bond, a peptide bond, and 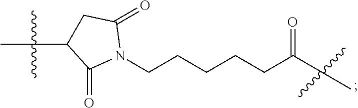 [SMILES: *C(=O)CCCCCN1C(=O)CC(*)C1=O]
preferably, the linker is selected from a group consisting of the disulfide bond, the aminoamide bond, the amide bond, and the 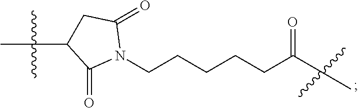 [SMILES: *C(=O)CCCCCN1C(=O)CC(*)C1=O]
preferably, the linker is selected from the

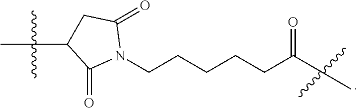 [SMILES: *C(=O)CCCCCN1C(=O)CC(*)C1=O]

4. The albumin conjugate, or the pharmaceutically acceptable salt or the solvate thereof according to any one of claims 1-3, wherein the albumin conjugate comprises a linker unit;
preferably, the linker unit is selected from a group consisting of an enzyme-cleavable linker unit and an enzyme-uncleavable linker unit;
preferably, the enzyme-cleavable linker unit is selected from a group consisting of a protease cleavable linker unit;
preferably, the protease cleavable linker unit comprises a combination of two or more selected from a group consisting of valine, citrulline, alanine, glycine, lysine, phenylalanine, glutamic acid, serine, and aspartic acid;
preferably, the protease cleavable linker unit comprises a combination of one or more selected from a group consisting of valine-citrullinedipeptide, valine-alaninedipeptide, and glycine-glycine-phenylalanine-glycinetetrapeptide;
preferably, the protease cleavable linker unit is selected from a group consisting of valine-citrulline-p-aminobenzyloxy (Val-Cit-PAB), valine-alanine-p-aminobenzyloxy (Val-Ala-PAB), glycine-glycine-phenylalanine-glycine (Gly-Gly-Phe-Gly), valine-citrulline-p-aminobenzyloxy (Val-Cit-PAB), valine-alanine-p-aminobenzyloxy (Val-Ala-PAB), and glycine-glycine-phenylalanine-glycine (Gly-Gly-Phe-Gly).

5. The albumin conjugate, or the pharmaceutically acceptable salt or the solvate thereof according to any one of claims 1-4, wherein the albumin conjugate has a structure as shown in Formula I:
HSA-(R-linker-linker unit-D)ₙ (Formula I)
wherein:
HSA is selected from a group consisting of:
a) a protein comprising an amino acid sequence as set forth in SEQ ID NO:1, preferably a protein of the amino acid sequence as set forth in SEQ ID NO:1;
b) a protein having substitution, deletion, addition or any combination thereof of one or more amino acid residues compared with SEQ ID NO:1, the substitution being conservative substitution; and
c) a protein having at least 80%, at least 85%, at least 90%, at least 91%, at least 92%, at least 93%, at least 94%, at least 95%, at least 96%, at least 97%, at least 98%, at least 99% or 100% sequence identity compared with SEQ ID NO:1;
R is selected from HcyTFAc;
D is selected from a toxin molecule;
wherein the HcyTFAc has a structure of 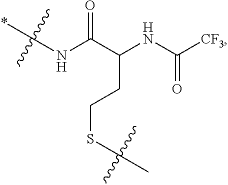
wherein * is a site directly linked to the albumin;
n is an integer selected from 1-16, preferably an integer selected from 2-8, more preferably an integer selected from 2-5.

6. The albumin conjugate, or the pharmaceutically acceptable salt or the solvate thereof according to claim 5, wherein the toxin molecule is selected from a group consisting of an antitubulin agent, maytansine, paclitaxel, camptothecin, duocarmycin, pyrrolobenzodiazepine (PBD), Eribulin, adriamycin, doxorubicin, gemcitabine, amethopterin, fluorouracil, taxol, bleomycin, mitomycin, cytarabine, ramycin, vinblastine, vincristine, morpholine-adriamycin, trifluorothymidine, Dxd, a nucleic acid, a nuclide, and a metal;
preferably, the toxin molecule is selected from a group consisting of the antitubulin agent, the maytansine, the paclitaxel, the camptothecin, the duocarmycin, the pyrrolobenzodiazepine (PBD), the Eribulin, the adriamycin, the irinotecan, the Dxd, the nucleic acid, the nuclide, and the metal;
preferably, the antitubulin agent is selected from a group consisting of a tubulin inhibitor, preferably selected from auristatin, more preferably selected from auristatin E, auristatin F, and auristatin D, most preferably selected from monomethyl auristatin E (MMAE), monomethyl auristatin F (MMAF), and monomethyl auristatin D (MMAD).

7. The albumin conjugate, or the pharmaceutically acceptable salt or the solvate thereof according to claim 5 or 6, wherein, preferably, the linker is selected from a group consisting of a thioether bond, a disulfide bond, an aminoamide bond, an amide bond, a peptide bond, and 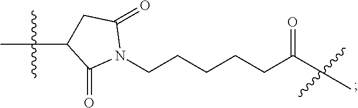 [SMILES: *C(=O)CCCCCN1C(=O)CC(*)C1=O]
preferably, the linker is selected from a group consisting of the disulfide bond, the aminoamide bond, the amide bond, and the 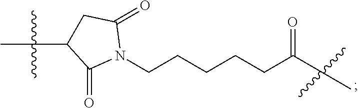 [SMILES: *C(=O)CCCCCN1C(=O)CC(*)C1=O]
preferably, the linker is selected from the

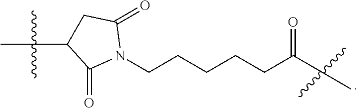 [SMILES: *C(=O)CCCCCN1C(=O)CC(*)C1=O]

8. The albumin conjugate, or the pharmaceutically acceptable salt or the solvate thereof according to any one of claims 5-7, wherein the linker unit is selected from a group consisting of an enzyme-cleavable linker unit and an enzyme-uncleavable linker unit;
preferably, the enzyme-cleavable linker unit is selected from a group consisting of a protease cleavable linker unit;
preferably, the protease cleavable linker unit comprises a combination of two or more selected from a group consisting of valine, citrulline, alanine, glycine, lysine, phenylalanine, glutamic acid, serine, and aspartic acid;
preferably, the protease cleavable linker unit comprises a combination of one or more selected from a group consisting of valine-citrullinedipeptide, valine-alaninedipeptide, and glycine-glycine-phenylalanine-glycinetetrapeptide;
preferably, the protease cleavable linker unit is selected from a group consisting of valine-citrulline-p-aminobenzyloxy (Val-Cit-PAB), valine-alanine-p-aminobenzyloxy (Val-Ala-PAB), glycine-glycine-phenylalanine-glycine (Gly-Gly-Phe-Gly), valine-citrulline-p-aminobenzyloxy (Val-Cit-PAB), valine-alanine-p-aminobenzyloxy (Val-Ala-PAB), and glycine-glycine-phenylalanine-glycine (Gly-Gly-Phe-Gly).

9. The albumin conjugate, or the pharmaceutically acceptable salt or the solvate thereof according to any one of claims 1-8, wherein the albumin conjugate is selected from a group consisting of conjugates below: 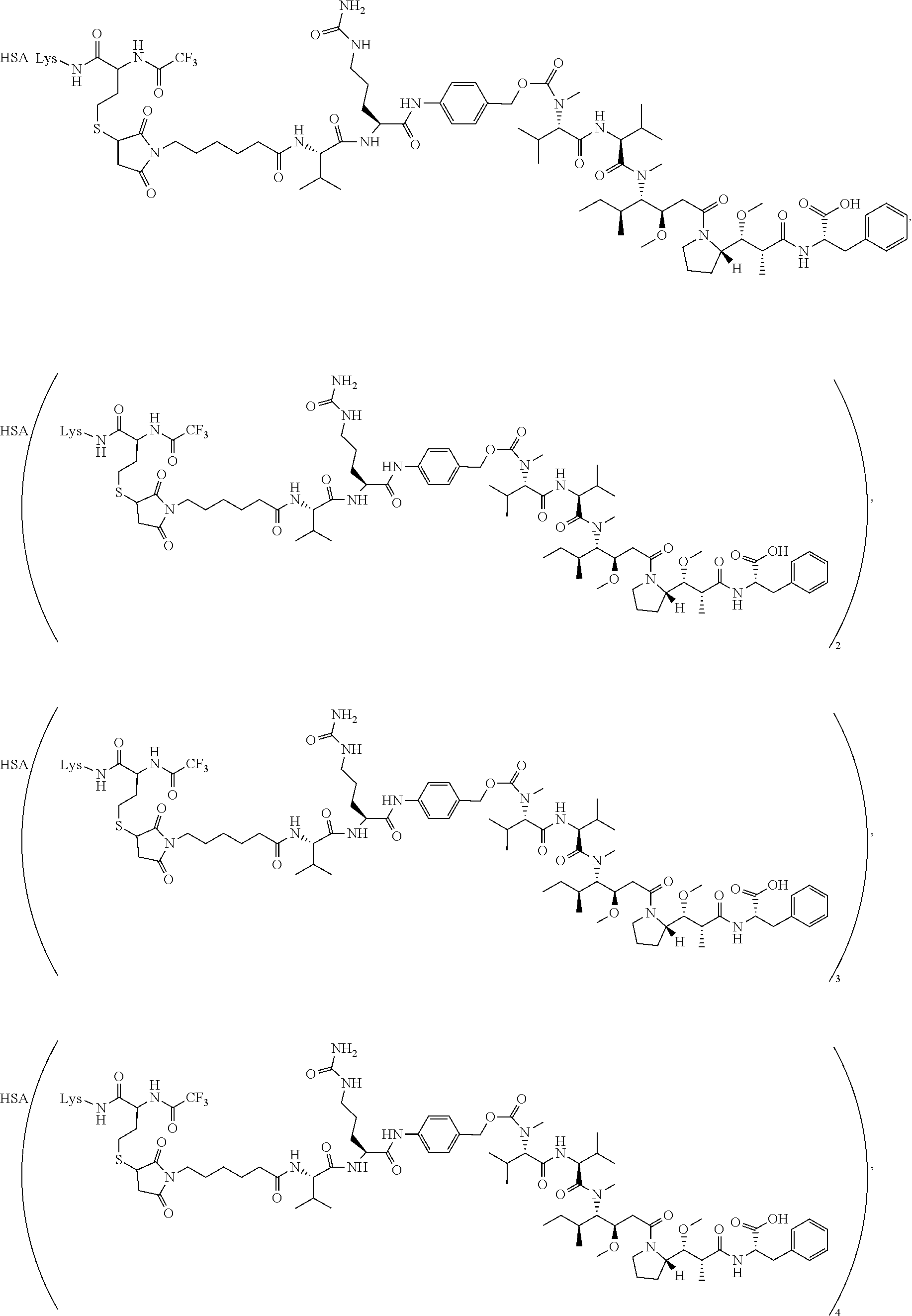 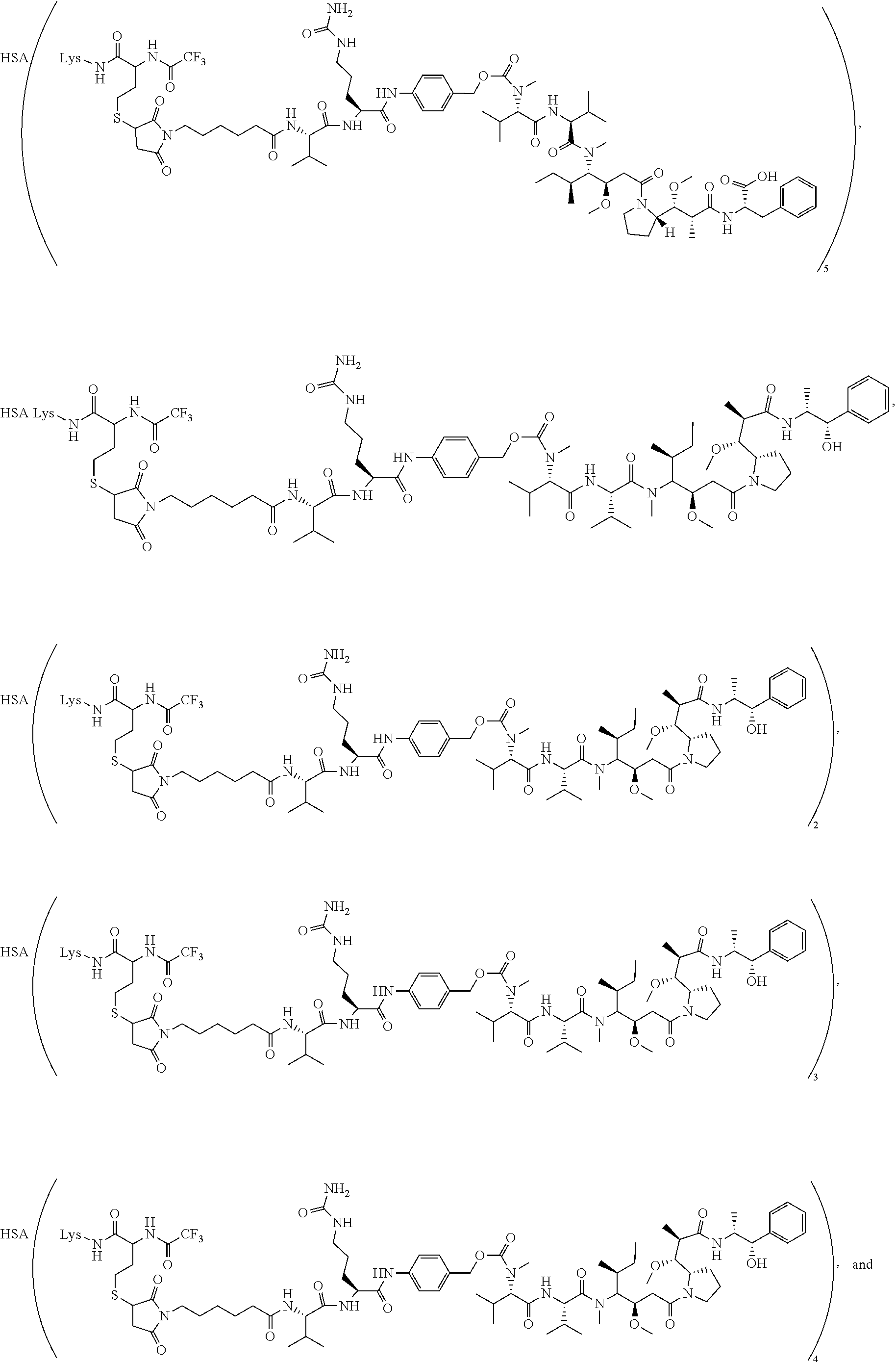 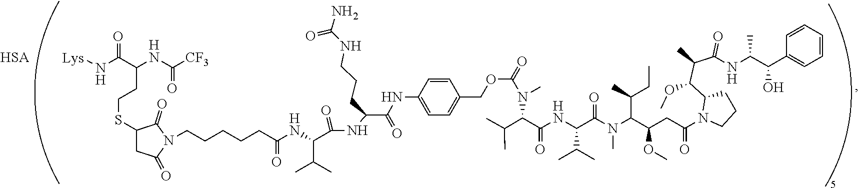
wherein Lys is a lysine residue in the amino acid sequence of the albumin.

10. A pharmaceutical composition, comprising a therapeutically effective amount of the albumin conjugate, or the pharmaceutically acceptable salt or the solvate thereof according to any one of claims 1-9, and one or more pharmaceutically acceptable carriers, diluents or excipients.

11. Use of the albumin conjugate, or the pharmaceutically acceptable salt or the solvate thereof according to any one of claims 1-9 or the composition according to claim 10 for preventing and/or treating a cancer;
preferably, the cancer is selected from a group consisting of breast cancer, ovarian cancer, cervical cancer, uterine cancer, prostate cancer, renal cancer, urinary system cancer, bladder cancer, liver cancer, pancreatic cancer, glioblastoma, lymphoma, gastric cancer, endometrial cancer, salivary gland cancer, esophageal cancer, lung cancer, colon cancer, rectal cancer, colorectal cancer, bone cancer, skin cancer, thyroid cancer, pancreatic cancer, brain tumor, melanoma, neuroglioma, neuroblastoma, glioblastoma multiforme, sarcoma, and leukemia,
